(19) United States
(12) Patent Application Publication
(10) Pub. No.: US 2020/0121821 A1
(43) Pub. Date: Apr. 23, 2020

(54) A DISPENSING SYSTEM FOR DISPENSING A FLUID INTO THE ATMOSPHERE

(71) Applicant: KANTERS SPECIAL PRODUCTS B.V., Lieshout (NL)

(72) Inventor: Pierluigi Salvi, Cesena (IT)

(21) Appl. No.: 16/627,864

(22) PCT Filed: Jun. 6, 2018
(86) PCT No.: PCT/NL2018/050366

§ 371 Date: Dec. 31, 2019

(30) Foreign Application Priority Data
Jul. 3, 2017 (NL) 2019162

(51) Int. Cl. A61L 9/12

(52) U.S. Cl. CPC A61L 9/127; A61L 2209/211; A61L 2209/13

(57) ABSTRACT

The invention relates to a dispensing system for dispensing a fluid into the atmosphere, wherein the system comprises a reservoir for containing a liquid solution, and a quick release coupling for manually connecting and disconnecting a first end of a capillary member outside the reservoir to an outlet of the reservoir. The quick release coupling provides a fluid connection between the interior of the reservoir and the capillary

member, such that the capillary member is able to draw by capillary attraction up the liquid solution from the reservoir from the first end connected to the quick release coupling towards its second end for dispensing a gaseous fluid into the atmosphere by the capillary member by means of evaporation of the liquid solution.

The invention relates to a dispensing system for dispensing a fluid into the atmosphere.

The invention also relates to a room equipped with a dispensing system for dispensing a fluid into the atmosphere.

The invention further relates to a method for dispensing a fluid into the atmosphere.

U.S. Pat. No. 5,868,998 discloses a method of fumigation which will destroy microorganisms in the environment of hatching and newly hatched poultry whereby a disinfecting solution such as hydrogen peroxide is applied into the environment by a micro-aerosol. This document teaches that a micro-aerosol dispensing apparatus has a construction for actively dispensing a gaseous fluid in the environment such as specialized pneumatic driven nozzles, thermal foggers, high velocity airstreams with a venturi nozzle or ultrasonic apparatus.

A drawback of the known dispensing apparatus is that it does not function autonomous, i.e. the dispensing apparatus requires electrical power to dispense the gaseous fluid for conditioning the environment. Installation of the known apparatus further requires the presence of a power supply in the hatcher. This also results in additional installation time for installing the apparatus in the hatcher. Further, the known apparatus comprises electrical components which have a certain lifetime, such that the apparatus requires maintenance.

It is a primary object of the present invention to provide an autonomous dispensing system for dispensing a fluid into the atmosphere. In addition, it is a secondary object of the invention to provide a user-friendly system for dispensing a fluid into the atmosphere that can be installed and maintained by any person without requiring installers such as electrician for installing and maintaining the electrical equipment and components.

This object is achieved by the system for dispensing a fluid into the atmosphere as claimed in claim 1.

The dispensing system comprises a reservoir for containing a liquid solution, and a quick release coupling for manually connecting and disconnecting a first end of a capillary member outside the reservoir to an outlet of the reservoir, wherein the quick release coupling provides a fluid connection between the interior of the reservoir and the capillary member, such that the capillary member is able to draw by capillary attraction up the liquid solution from the reservoir from the first end connected to the quick release coupling towards its second end for dispensing a gaseous fluid into the atmosphere by the capillary member by means of evaporation.

The dispensing system conditions the atmosphere/environment by dispensing the gaseous fluid into the atmosphere/environment by the capillary member by means of evaporation. By means of the gaseous fluid the conditions in the environment of the dispensing system can be optimized for a certain goal. For example, it is possible to increase the comfort or the hygiene by means of the gaseous fluid. In particular, it is possible to use the dispensing system to reduce microorganism contamination of the environment inside an incubator. The dispensing system can advantageously be used without electrical power, because the liquid solution diffuses into the capillary member due to capillary action, wherein evaporation can take place throughout the capillary member. By means of the capillary member it is possible to increase the interface between the liquid solution and the atmosphere surrounding the liquid solution. This increased interface facilitates the evaporation process of the liquid solution into a gas to be dispensed to the atmosphere. Hence, an autonomous, i.e. power independent, dispensing system is provided that can be installed by anyone. Further, the system does not require any or only minimal maintenance.

As a result of the minimal structural components of the system it is further possible to provide a disposable system, i.e. a single use system such that once the system has been used for a certain time period, for example a week, in for example an incubator, it can be replaced by a complete new system.

The quick release coupling and/or the capillary member make it possible that the system functions continuously even during the replacement of an empty reservoir with a new reservoir, because the replacement can be done within a few seconds (for example 30-60 seconds) time by anyone without the risk that in such a time period the capillary member runs dry of liquid solution to be dispensed to the atmosphere. In addition, it is possible by means of the quick release coupling to provide a system that is user-friendly.

Cotton, gauze, fibrous substances, foam, sponges and other types of connective material which cause a capillary action can be utilized to produce a capillary member.

The system may further comprise a conduit for transporting the liquid solution having a first end section connected to the outlet of the reservoir and having a second end section provided with or connected to the quick release coupling. Such a conduit has the advantage that the distance between the reservoir and the capillary member can be increased. This increased distance makes it possible that the reservoir can be positioned in a different atmosphere than at least a portion of the capillary member, such that the reservoir can be changed without entering the atmosphere to be conditioned by means of the gaseous fluid. Further, the conduit may serve as an additional back-up reservoir which prevents that the capillary member runs dry, for example during the replacement of the reservoir.

The system may comprise a flow regulator for controlling the flow of liquid solution to the capillary member. In this way it is possible to control and adapt the flow and as a result control the amount of gaseous fluid to be dispensed in the atmosphere.

The capillary member may be a wick, i.e. a bundle of fibers or a loosely twisted, braided, or woven cord, tape, or tube for example of soft spun cotton threads that by capillary attraction draws up the liquid solution from the reservoir. The wick may have a length between 0.01-5.0 m, preferably between 0.25-2.5 m. The cross section of the wick may a diameter between 0.1-5 cm. The word diameter may suggest that the cross section of the wick is circular, but the cross section of the wick may have any shape.

The quick release coupling may have a male part and a female part connected or to be connected to the capillary member, wherein the male part can be manually detachably connected in the female part and/or disconnected from the second part. In this way a user-friendly coupling is provided that can be used by anyone to change for example a reservoir in a quick and efficient manner.

The system may further comprise a frame structure for carrying the reservoir, wherein the reservoir is located by means of the frame structure at a higher position seen from the underground than a major portion of the capillary member.

Such a frame introduces a gravity flow provided by the height differences between the reservoir and the capillary member which may for example facilitate transport of the liquid solution throughout the capillary member.

Further, it is possible that a portion of the capillary member comprises an outer sleeve (not shown) of material impermeable to the liquid solution which sleeve surrounds the capillary member. The sleeve makes it possible to allow the evaporation to take place in the desired capillary member section(s). Further, it is possible to position the reservoir in a different environment than the portion of the capillary member without the outer sleeve.

The liquid solution can be a disinfecting solution comprising antimicrobial agents, preferably containing hydrogen peroxide, more preferred the liquid solution comprises 3-70% by weight of hydrogen peroxide, still more preferred the liquid solution comprises 40-55% by weight of hydrogen peroxide.

The liquid solution containing hydrogen peroxide can be used for different applications, wherein each application may have has its preferred composition of the liquid solution containing hydrogen peroxide. In an incubator the liquid solution preferably comprises 40-55% by weight of hydrogen peroxide, because than a minimal volume of the liquid solution is required to obtain a maximal disinfecting effect in the incubator.

The liquid solution may be a volatile liquid solution. Volatile liquid solutions evaporate at normal room temperatures. Volatile liquid solutions in this document are liquid solutions having a boiling temperature between 15 and 150 degrees Celsius, preferably between 20 and 125 degrees Celsius.

The invention also relates to a room equipped with a dispensing system for dispensing a fluid into the atmosphere as described in this document. An advantage of the system is that the reservoir can be positioned outside the room, whereas at least the second end of the capillary member can be positioned inside the room for dispensing a gaseous fluid into the atmosphere of the room. Such a configuration of the system with respect to a room makes it possible that the reservoir can be changed without entering the room which also prevents or reduces the risk that the atmosphere in the room is contaminated from the outside. This aspect is in particular an advantage if the room is a clean room or an incubator (hatcher), wherein it is preferred that the influences outside the room have zero or minimal impact on the atmosphere inside the room.

The invention further relates to a method for dispensing a fluid into the atmosphere. The method uses a reservoir containing a liquid solution, and a quick release coupling for manually connecting and disconnecting a first end of a capillary member outside the reservoir to an outlet of the reservoir, wherein the quick release coupling provides a fluid connection between the interior of the reservoir and the capillary member, such that the capillary member draws by capillary attraction up the liquid solution from the reservoir from the first end connected to the quick release coupling towards its second end to dispense a gaseous fluid into the atmosphere by the capillary member by means of evaporation of the liquid solution.

The method has the same advantages as described for the system above. Further, the method can be implemented in a relatively simple manner, even in an environment without the presence of any power supplies.

The liquid solution may evaporate as a disinfecting gas into the atmosphere, wherein the atmosphere is the atmosphere in a room, for example a cooling unit (refrigerator), a clean room or an incubator. The method makes it possible to provide an atmosphere having between 50-300 ppm liquid solution from the reservoir per cubic meter.

The invention will now be explained in more detail on the basis of exemplary embodiments in the appended drawings, in which:

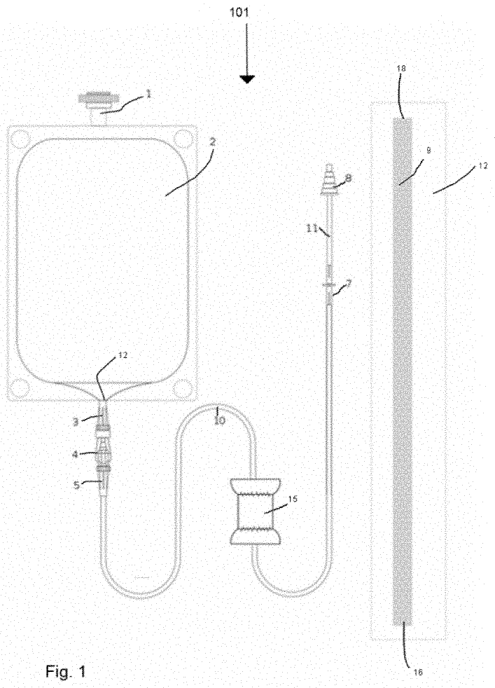

FIG. 1 shows a schematic view of a first embodiment of the components of a system for dispensing a fluid into the atmosphere;

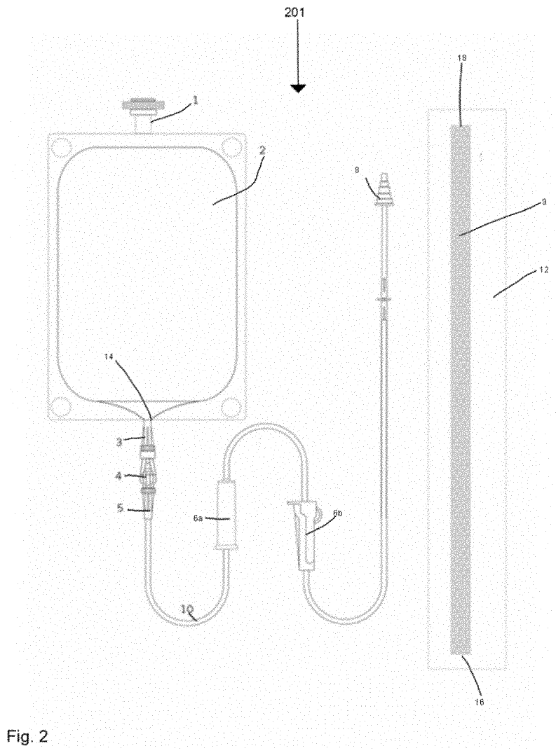

FIG. 2 shows a schematic view of a second embodiment of the components of a system for dispensing a fluid into the atmosphere.

Like parts are indicated by the same numerals in the figures.

FIGS. 1 and 2 show two embodiments of components to produce a system 101; 201 for dispensing a fluid into the atmosphere.

Each system 101; 201 comprises a reservoir 2 for containing a liquid solution, and a quick release coupling 8 for manually connecting and disconnecting a first end 16 of a capillary member 9 outside the reservoir 2 to an outlet 14 of the reservoir 2. The reservoir 2 is an intravenous (IV) bag with a volume of 1.5 litre, wherein the reservoir 2 is an atoxic PVC bag.

The quick release coupling 8 is in the embodiments shown a multi-caliber connector which provides a fluid connection between the interior of the reservoir 2 and the capillary member 9.

The capillary member 9 is a wick. The length of the wick may vary between 0.01 m and 5 m, normally the length is 0.5-2.0 m. The wick 9 shown in the figures is packed in a sterile packing 12 before use. After unpacking the wick 9 from the sterile packing 12, a user may connect the first end 16 of the wick 9 to the multi-caliber connector 8.

A first end 16 of the wick 9 can be hollow or can be or is provided with a blind hole (not shown) to receive the multi-caliber connector 8. However, it is also possible that the quick release coupling (not shown) has a female external part (not shown) connected or to be connected to the wick 9, wherein the multi-caliber connector 8 (the male part) can be manually detachably connected in the female part and/or disconnected from the female part. It is also possible that the male part has a different configuration than the caliber connector 8 shown in the figures.

After providing a fluid connection between the interior of the reservoir 2 and the wick 9, the wick 9 is able to draw by capillary attraction up the liquid solution from the reservoir 2 from the first end 16 connected to the quick release coupling towards its second end 18. Then, the liquid solution in the wick 9 will be dispensed as a gaseous fluid into the atmosphere by means of evaporation of the liquid solution.

The system 101; 102 shown in FIGS. 1 and 2 further comprise a first conduit section 10 and a second conduit section 11 which are connected to each other by means of a connector 7. The first conduit section 10 is connected to the outlet 14 of the reservoir 2 by means of a female luer lock 3, a needle free port, 4 and a male leur lock 5.

The system 101 (FIG. 1) differs from the system 102 (FIG. 2) in that the first conduit section 10 shown in FIG. 1 comprises an flow regulator 15 with a range of 5-250 ml/h and the first conduit section 10 of the system 102 shown in FIG. 2 comprises a drip chamber 6a and a roller clamp 6b providing control of flow rate of the liquid solution of the reservoir 2 toward the wick 9.

The system 101; 102 further comprises a frame structure (not shown) for carrying the reservoir 2 by means of a suspension point 1, wherein the reservoir 2 is in use located by means of the frame structure at a higher position seen from the underground than a major portion of the wick 9. Such a frame is able to introduce a gravity flow provided by the height difference between the reservoir 2 and the wick 9 in use which facilitates the transport of the liquid solution throughout the wick 9.

The system 101; 102 can be used in an incubator room (not shown) to provide continuous protection for commercially raised poultry before the time the birds first break through the egg shell until the birds are removed from the incubator. Further, it is possible with the system and method as defined in the claims to decrease the microbial contamination in the atmosphere of the incubator without adversely affecting the safety of the birds or of the people working in or in the presence of the incubators. The system and method may use a liquid solution sold under the name Aqua-Clean by Kanters Special Products B.V. which is a disinfecting solution comprising antimicrobial agents, in particular the solution comprises 40-55% by weight of hydrogen peroxide. The advantages of Aqua-clean are that it is 100% bio-degradable, does not contain chlorine and/or bromine and that drainage is possible without neutralisation because hydrogen peroxide dissolves in water and oxygen.

After installing the system 101; 102 in an incubator, the system is able to continuously non-stop dispense a gaseous disinfecting fluid into the atmosphere of the incubator. In this process the reservoir 2 of 1.5 litre can be used up to two days after which the reservoir will have to be replaced by a new reservoir 2. The replacement can be done be anyone in a few seconds time and during the replacement of the reservoir 2 the wick 9 will continue to dispense gaseous disinfecting fluid into the atmosphere of the incubator without interruption. In an incubator the ventilation of air varies between 300-700 cubic metres per hour. The system is able to provide an atmosphere having between 50-300 ppm liquid solution from the reservoir per cubic meter.

The quick release coupling (not shown) can also be provided with a valve to prevent leakage of the liquid solution during replacement of the wick 9 and/or the reservoir 2.

1. A dispensing system for dispensing a fluid into the atmosphere, wherein the system comprises a reservoir for containing a liquid solution, and a quick release coupling for manually connecting and disconnecting a first end of a capillary member outside the reservoir to an outlet of the reservoir, wherein the quick release coupling provides a fluid connection between the interior of the reservoir and the capillary member, such that the capillary member is able to draw by capillary attraction up the liquid solution from the reservoir from the first end connected to the quick release coupling towards its second end for dispensing a gaseous fluid into the atmosphere by the capillary member by means of evaporation of the liquid solution.

2. The dispensing system according to claim 1, wherein the system comprises a conduit for transporting the liquid solution having a first end section connected to the outlet of the reservoir and having a second end section provided with or connected to the quick release coupling.

3. The dispensing system according to claim 1, wherein the system comprises a flow regulator for controlling the flow of the liquid solution to the capillary member.

4. The dispensing system according to claim 1, wherein the capillary member is a wick.

5. The dispensing system according to claim 1, wherein the quick release coupling has a male part and a female part connected or to be connected to the capillary member, wherein the male part can be manually detachably connected in the female part and/or disconnected from the female part.

6. The dispensing system according to claim 1, wherein the system comprises a frame structure for carrying the reservoir, wherein the reservoir is located by means of the frame structure at a higher position seen from the underground than a major portion of the capillary member.

7. The dispensing system according to claim 1, wherein a portion of the capillary member comprises an outer sleeve of material impervious to the liquid solution, surrounding the capillary member.

8. The dispensing system according to claim 1, wherein the liquid solution is a volatile liquid solution.

9. The dispensing system according to claim 1, wherein the liquid solution is a disinfecting solution comprising antimicrobial agents.

10. The dispensing system according to claim 1, wherein the reservoir is a bag, preferably an intravenous (IV) bag.

11. (canceled)

12. (canceled)

13. A method for dispensing a fluid into the atmosphere, wherein the method uses a reservoir containing a liquid solution, and a quick release coupling for manually connecting and disconnecting a first end of a capillary member outside the reservoir to an outlet of the reservoir, wherein the quick release coupling provides a fluid connection between the interior of the reservoir and the capillary member, such that the capillary member draws by capillary attraction up the liquid solution from the reservoir from the first end connected to the quick release coupling towards its second end to dispense a gaseous fluid into the atmosphere by the capillary member by means of evaporation of the liquid solution.

14. The method according to claim 13, wherein the method dispenses 10-2500 ml liquid solution per hour into the atmosphere.

15. The method according to claim 13, wherein the liquid solution evaporates as a disinfecting gas into the atmosphere.

16. The method according to claim 13, wherein the method provides an atmosphere having between 50-300 ppm liquid solution from the reservoir per cubic meter.

17. The method according to claim 13, wherein the liquid solution is a disinfecting solution comprising antimicrobial agents.

18. The method according to claim 17, wherein the liquid solution is Aqua-Clean.
